(19) United States
(12) Patent Application Publication
(10) Pub. No.: US 2023/0037041 A1
(43) Pub. Date: Feb. 2, 2023

(54) APPARATUS AND METHOD FOR PREPARING NUCLEIC ACID SEQUENCES USING ENZYME

(71) Applicant: YUANDIAN BIOLABS CO., LTD., Hsinchu (TW)

(72) Inventors: Cheng-Yao Chen, Hsinchu (TW); Jui-Kang Yen, Hsinchu (TW)

(21) Appl. No.: 17/789,527

(22) PCT Filed: Dec. 30, 2019
(86) PCT No.: PCT/US2019/068864

§ 371 Date: Jun. 28, 2022

(51) Int. Cl. B01J 19/00; C12P 19/34; C12N 9/12

(52) U.S. Cl. CPC B01J 19/0046; B01J 19/0013; B01J 19/0053; C12N 9/1252; C12P 19/34; C12Y 207/07007

(57) ABSTRACT

An apparatus for preparing nucleic acid sequences using an enzyme, including a reactor, a plurality of nucleotide material bottles, a deblocking material bottle, and a liquid delivering device. The reactor includes a reaction substrate having a pretreated surface. Each of the nucleotide material bottles is adapted to contain a first reaction solution, and the first reaction solution includes a reaction enzyme and a nucleotide having a terminal protecting group. The deblocking material bottle is adapted to contain a deblocking solution. The liquid delivering device is connected to the reactor, the nucleotide material bottles and the deblocking material bottle. The reaction enzyme is adapted to dispose the nucleotide having the terminal protecting group on the pretreated surface. The reactor has an operating temperature of 45° C.-105° C. A method for preparing nucleic acid sequences using an enzyme of the invention is provided.

### FIELD OF THE INVENTION

The present invention relates to an apparatus and a method for preparing a biomolecular structure, and more particularly to an apparatus and a method for preparing nucleic acid sequences using an enzyme.

### BACKGROUND OF THE INVENTION

In the mid-20th century, there were several key breakthroughs in the fields of genetics and biochemistry that have led us to current day medicine. This was a cascade of events that started with X-ray-induced gene knockout studies in 1941, which made the connection that genes were directly involved in enzyme function. It soon followed that genes themselves were comprised of nucleic acids (DNA), and a double helix was an orderly structure of nucleic acids that stored genetic information and could be precisely replicated by a DNA polymerase.

Nucleic acid synthesis is vital to modem biotechnology. The rapid pace of development in the biotechnology arena has been made possible by the scientific community’s ability to artificially synthesis DNA, RNA and proteins. Artificial DNA synthesis, a 1 billion and growing market, allows biotechnology and pharmaceutical companies to develop a range of peptide therapeutics, such as insulin for the treatment of diabetes. It allows researchers to characterize cellular proteins to develop new small molecule therapies for the treatment of diseases our aging population faces today, such as heart disease and cancer.

However, current DNA synthesis technology does not meet the demands of the biotechnology industry. While the benefits of DNA synthesis are numerous, an oft-mentioned problem prevents the further growth of the artificial DNA synthesis industry, and thus the biotechnology field. Despite being a mature technology, it is practically very hard to synthesis a DNA strand greater than 200 nucleotides in length, and most DNA synthesis companies only offer up to 120 nucleotides. In comparison, an average protein-coding gene is of the order of 2000 - 3000 nucleotides, and an average eukaryotic genome numbers in the billions of nucleotides. Thus, all major gene synthesis companies today rely on variations of a “synthesis and stitch” technique, where overlapping 40-60-mer fragments are synthesized and stitched together by PCR (see Young, L. et al. (2004) Nucleic Acid Res.32, e59). Current methods offered by the gene synthesis industry generally allow up to 3 kb in length for routine production.

To date, there are two main categories of in vitro synthesis of nucleic acids: chemical synthesis or enzymatic synthesis. The most common in vitro nucleic acid chemical synthesis method is the phosphoramidite polymerization method described by Adams et al. (1983, J. Amer. Chem Soc., 105: 661) and Froehler et al. (1983, Tetrahedron Lett, 24: 3171). In this method, each nudeotide to be added is protected at the level of the 5'-OH group so as to avoid an uncontrolled polymerization of several nucleotides of the same type. Generally the protection of the 5'-OH group is carried out by a trityl group. In order to avoid possible degradation due to the use of powerful reagents, the bases carried by the nucleotides can also be protected. Generally the protection used involves an isobutyryl group (Reddy et al., 1997, Nucleosides & Nucleotides, 16: 1589). After each incorporation of new nucleotides, the 5'-OH group of the last nucleotide of the chain undergoes a deblocking reaction in order to make it available for the next polymerization step. The nitrogenous bases carried by the nucleotides composing the nucleic acid, they are deblocked only after completion of the complete polymerization.

Chemical synthesis methods, such as those discussed above, require large amounts of unstable, hazardous, expensive reagents that can impact the environment and health. The devices making it possible to carry out these syntheses in a practical way are complex, require a large investment and must be operated by a qualified and dedicated workforce. One of the major disadvantages of these chemical synthesis techniques lies in their low yield. During each cycle, the coupling reaction occurs only in 98 to 99.5% of the cases, leaving in the reaction medium nucleic acids that do not have a correct sequence. As the synthesis progresses, the reaction medium is greatly enriched in fragments with a totally incorrect sequence. The deletion type errors that occur thus have particularly dramatic repercussions causing a shift in the reading frame of the nucleic acid fragments considered.

Thus, for a correct coupling reaction in 99% of cases, a nucleic acid comprising 70 nucleotides will be synthesized with a yield of less than 50%. Which means that after 70 cycles of addition, the reaction medium will comprise more fragments with a wrong sequence than fragments with a correct sequence. This mixture is then unsuitable for further use.

The methods of chemical synthesis of nucleic acid are therefore ineffective for the synthesis of long fragments because they generate a very large amount of fragments having an incorrect sequence, then considered as impurities. In practice, the maximum length of fragments that can be efficiently produced by these methods is between 50 and 100 nucleotides.

On the other hand, the difference between the methods of enzyme synthesis and chemical synthesis is that the method of enzyme synthesis uses an enzymatic catalyst for carrying out the coupling step.

U.S. Pat. No. 7070440 B1 discloses a method of performing DNA synthesis using a template-dependent polymerase. However, this technology is not suitable for de novo nucleic acid synthesis because of the requirement for an existing nucleic acid strand to act as a template.

Enzymes that can carry out the coupling reaction between nucleotides in the absence of a template strand are needed. DNA polymerase is one of the enzymes that can achieve the conditions. DNA polymerases have been categorized in seven evolutionary families based on their amino acid sequences: A, B, C, D, X, Y, and RT. The families of DNA polymerases appear to be unrelated, i.e., members of one family are not homologous to members of any other family. A DNA polymerase is determined to be a member of given family by its homology to a prototypical member of that family. For example, members of family A are homologous to E. coli DNA polymerase I; members of family B are homologous to E. coli DNA polymerase II; members of family C are homologous to E. coli DNA polymerase III; members of family D are homologous to Pyrococcus furiosus DNA polymerase; members of family X are homologous to eukaryotic DNA polymerase beta; members of family Y are homologous to eukaryotic RAD30; and members of family RT are homologous to reverse transcriptase.

Various attempts have been made to use a terminal deoxynucleotidyl transferase for controlled de novo single stranded DNA synthesis (Ud-Dean et al. (2009) Syst. Synth. Boil., 2, 67-73, US 5763594 and US 8808989). Uncontrolled de novo single stranded DNA synthesis, as opposed to controlled, takes advantage of TdT’s deoxynucleotide triphosphate (dNTP) 3' tailing properties on single-stranded DNA to create, for example, homopolymeric adaptor sequences for next generation sequencing library preparation (Roychoudhur R. et al. (1976) Nucleic Acids Res. 3, 10-116 and WO 2003/050242).

However, as its key purpose is to increase antigen receptor diversity, terminal deoxynucleotidyl transferase may resist normal, predictable behavior observed with most replicative, high-fidelity polymerases. These represent many challenges to its use in the nucleotide synthesis cycle for high-throughput automation. Although many documents (WO 2016128731A1, WO 2018217689A1, WO 2019135007A1) have disclosed the use of modified terminal deoxynucleotidyl transferase for enzyme synthesis, they still have a limit on the rate of reaction, and still increase the probability of error when performing large quantities of synthesis of nucleic acid sequences.

In summary, the chemical synthesis of nucleic acids can synthesize nucleic acids in large quantities. However, the reagents used are too expensive and pollute the environment, and the probability of error in the synthesis process is higher. The enzymatic synthesis of nucleic acids is inexpensive, and the nucleic acid sequences have a higher degree of accuracy. However, due to the reaction rate of the enzyme, large quantities of synthesis cannot be performed. Therefore, there is still no better way to synthesize nucleic acids today, which can solve the problem of correct and large quantities of synthesis of nucleic acids.

### SUMMARY OF THE INVENTION

The present invention provides an apparatus for preparing nucleic acid sequences using an enzyme, which increases the efficiency of preparing nucleic acid sequences.

The present invention provides a method for preparing nucleic acid sequences using an enzyme, which increases the efficiency of preparing nucleic acid sequences.

An apparatus for preparing nucleic acid sequences using an enzyme provided in an embodiment of the invention includes a reactor, a plurality of nucleotide material bottles, a deblocking material bottle, and a liquid delivering device. The reactor includes a reaction substrate having a pretreated surface. Each of the nucleotide material bottles is adapted to contain a first reaction solution, and the first reaction solution includes a reaction enzyme and a nucleotide having a terminal protecting group. The deblocking material bottle is adapted to contain a deblocking solution. The liquid delivering device is connected to the reactor, the nucleotide material bottles and the deblocking material bottle, and is adapted to deliver the first reaction solution and the deblocking solution to the pretreated surface. The reaction enzyme is adapted to dispose the nucleotide having the terminal protecting group on the pretreated surface. The deblocking solution is adapted to remove the terminal protecting group of the nucleotide. The reactor has an operating temperature of 45° C. - 105° C.

In one embodiment of the invention, the reactor has the operating temperature of 50° C. - 85° C. .

In one embodiment of the invention, the reactor has the operating temperature of 55° C. - 75° C.

In one embodiment of the invention, the reaction enzyme is a DNA polymerase.

In one embodiment of the invention, the reaction enzyme is family A DNA polymerase.

In one embodiment of the invention, the reaction enzyme is family B DNA polymerase.

In one embodiment of the invention, the reaction enzyme is family X DNA polymerase.

In one embodiment of the invention, the liquid delivering device includes an extraction device adapted to extract the first reaction solution and the deblocking solution to the pretreated surface.

In one embodiment of the invention, the extraction device is a syringe pump or an air pressure valve.

In one embodiment of the invention, the pretreated surface has a plurality of primers adapted to couple the nucleotide having the terminal protecting group.

In one embodiment of the invention, the deblocking solution includes a reducing agent or a deblocking enzyme.

In one embodiment of the invention, the apparatus for preparing nucleic acid sequences using an enzyme further including a restriction enzyme material bottle, adapted to contain a second reaction solution including a restriction enzyme, wherein the liquid delivering device is further connected to the restriction enzyme material bottle to deliver the second reaction solution to the pretreated surface.

In one embodiment of the invention, the apparatus for preparing nucleic acid sequences using an enzyme further including a heater, connected to the reactor and adapted to heat the reactor.

In one embodiment of the invention, the liquid delivering device includes a first switch valve and a plurality of first delivering tubes, the first delivering tubes are connected between the nucleotide material bottles and the first switch valve and between the deblocking material bottle and the first switch valve, the first switching valve is connected to the reactor and adapted to switch between the first delivering tubes.

In one embodiment of the invention, the apparatus for preparing nucleic acid sequences using an enzyme further including a waste bottle and a product bottle, wherein the liquid delivering device includes a second switch valve and a plurality of second delivering tubes, the second delivering tubes are connected between the waste bottle and the second switch valve and between the product bottle and the second switch valve, the second switching valve is connected to the reactor and adapted to switch between the second delivering tubes.

In one embodiment of the invention, the apparatus for preparing nucleic acid sequences using an enzyme further including a power supply element, adapted to supply power to the liquid delivering device.

A method for preparing nucleic acid sequences using an enzyme provided in an embodiment of the invention includes: (1) providing a reaction substrate having a pretreated surface. (2) Disposing a nucleotide having a terminal protecting group on the pretreated surface by a reaction enzyme, and an operating temperature is 45° C. - 105° C. (3) Removing the terminal protecting group of the nucleotide by a deblocking solution. (4) Coupling another nucleotide having the terminal protecting group to the nucleotide by the reaction enzyme, and an operating temperature is 45° C. - 105° C. (5) Determining whether a nucleic acid sequence is completed, and if so, obtaining the nucleic acid sequence, if otherwise repeating steps (3) and (4).

In one embodiment of the invention, the method of disposing a nucleotide having a terminal protecting group on the pretreated surface includes disposing a first reaction solution on the pretreated surface, in which the first reaction solution includes the reaction enzyme and a nucleotide having the terminal protecting group.

In one embodiment of the invention, the method of disposing the first reaction solution on the pretreated surface includes delivering the first reaction solution to the pretreated surface by a liquid delivering device.

In one embodiment of the invention, the method of adjusting the operating temperature to 45° C. - 105° C. includes heating the reaction substrate by a heater.

In one embodiment of the invention, the pretreated surface has a plurality of primers, the method of disposing a nucleotide having a terminal protecting group on the pretreated surface comprises coupling the nucleotide having the terminal protecting group to the primers by the reaction enzyme.

In one embodiment of the invention, the method for preparing nucleic acid sequences using an enzyme further including: (6) cutting the nucleic acid sequence from the pretreated surface by restriction enzyme.

Since the apparatus and the method for preparing nucleic acid sequences using an enzyme of the embodiment of the invention use an enzyme to synthesize a nucleic acid sequence, it is less likely to pollute the environment and may reduce the cost compared to the method of chemical synthesis. In addition, the reactor has an operating temperature of 45° C. -105° C., compared to the conventional use of enzyme at 37° C., the embodiment of the invention may exhibit better activity by using an enzyme at 45° C. or higher, and the synthesis of a plurality of nucleic acid sequences may be carried out simultaneously with the apparatus for preparing nucleic acid sequences using an enzyme of the invention, thereby increasing the efficiency of preparing nucleic acid sequences.

Other objectives, features and advantages of The invention will be further understood from the further technological features disclosed by the embodiments of The invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic block diagram of an apparatus for preparing nucleic acid sequences using an enzyme of one embodiment of the invention;

FIG. 2 is a schematic diagram of an apparatus for preparing nucleic acid sequences using an enzyme of one embodiment of the invention;

FIG. 3 is a schematic diagram of a combination of a reactor and a heater of one embodiment of the invention;

FIG. 4 is a schematic diagram of a combination of a reactor and a heater of another embodiment of the invention;

FIG. 5 is a schematic diagram of an apparatus for preparing nucleic acid sequences using an enzyme of another embodiment of the invention;

FIG. 6 is a schematic flow diagram of a method for preparing nucleic acid sequences using an enzyme of one embodiment of the invention; and

FIGS. 7A, 7B, 7C, 7D and 7E are schematic diagrams of a reaction of synthesizing nucleic acid sequences of one embodiment of the invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

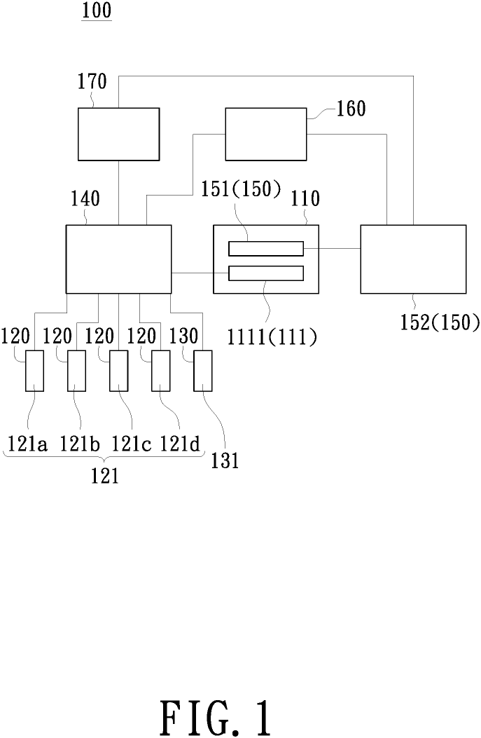

FIG. 1 is a schematic block diagram of an apparatus for preparing nucleic acid sequences using an enzyme of one embodiment of the invention. Referring to FIG. 1, an apparatus for preparing nucleic acid sequences using an enzyme 100 of the embodiment includes a reactor 110, a plurality of nucleotide material bottles 120, a deblocking material bottle 130, and a liquid delivering device 140. The reactor 110 includes a reaction substrate 111 having a pretreated surface 1111. Each of the nucleotide material bottles 120 is adapted to contain a first reaction solution 121, and the first reaction solution 121 includes a reaction enzyme and a nucleotide having a terminal protecting group. The deblocking material bottle 130 is adapted to contain a deblocking solution 131. The liquid delivering device 140 is connected to the reactor 110, the nucleotide material bottles 120 and the deblocking material bottle 130, and is adapted to deliver the first reaction solution 121 and the deblocking solution 131 to the pretreated surface 1111.

A material of the reaction substrate 111 includes, for example, silicon, glass (SiO₂), a metal or a polymer such as polycarbonate or polymethyl methacrylate, but is not limited thereto. The reaction substrate 111 is, for example, a plate-like structure or a three-dimensional structure, and the pretreated surface 1111 is a plane of the plate-like structure or an integral surface of the three-dimensional structure. For example, the pretreated surface 1111 of the three-dimensional structure may be a spherical surface of superparamagnetic beads (SPMB) or a porous surface of a CPG (controlled pore glass) Frit.

The quantity of the nucleotide material bottles 120 in FIG. 1 is exemplified by four, but the quantity is not limited thereto. Taking a synthesis of single-stranded deoxyribonucleic acid (ssDNA) sequences as an example, the nitrogenous bases of the nucleotides required for their synthesis can be further divided into four categories: adenine (A), thymine (T), cytosine (C), guano (G). Therefore, the first reaction solution 121 in the four nucleotide material bottles 120 corresponds to the four different types of nucleotides (dAMP, dTMP, dCMP, dGMP), respectively, that is, it is divided into the first reaction solutions 121a, 121b, 121c, 121d. When synthesizing a ribonucleic acid (RNA) sequence, a material of a uracil nucleotide (UMP) is also required. Depending on the design requirements, more than one of the nucleotide material bottles 120 may be used for each type of nucleotide, or only one, two or three of four different types of nucleotides may be used. Therefore, the invention does not limit the quantity of the nucleotide material bottles 120.

As used herein, the terms “reaction enzyme”, “nucleotide having terminal protecting group”, “nucleotide”, “restriction enzyme” and the like described throughout the present invention shall be regarded as a general term for these substances, not the actual quantity thereof. For example, the quantity of the reaction enzyme of the first reaction solution is plural, and the quantity of the nucleotide having the terminal protecting group is also plural.

The apparatus for preparing nucleic acid sequences using an enzyme 100 further includes, for example, a heater 150, connected to the reactor 110 and adapted to heat the reactor 110. The reactor 110 has an operating temperature of 45° C. - 105° C., preferably 50° C. - 85° C., more preferably 55° C. - 75° C. The heater 150 includes, for example, a heating element 151 and a heating control element 152. The heating element 151 is disposed, for example, in the reactor 110 to heat the reactor 110, and the heating control element 152 is connected to the heating element 151, but is not limited thereto.

The apparatus for preparing nucleic acid sequences using an enzyme 100 further includes, for example, a power supply element 160 and a control module 170. The power supply element 160 is adapted to supply power to the liquid delivering device 140 and the heater 150. The control module 170 is electrically connected to the liquid delivering device 140 and the heater 150, and is adapted to allow the liquid delivering device 140 to select different nucleotide material bottles 120 or the deblocking material bottle 130, and control a heating temperature of the heater 150. The control module 170 is, for example, an electronic device that can achieve a control function, such as a tablet computer, a desktop computer and the like. The specific implementation of the apparatus for preparing nucleic acid sequences using an enzyme 100 will be further described below with reference to the drawings, but the specific architecture of the apparatus for preparing nucleic acid sequences using an enzyme 100 of the invention is not limited to the embodiments listed below.

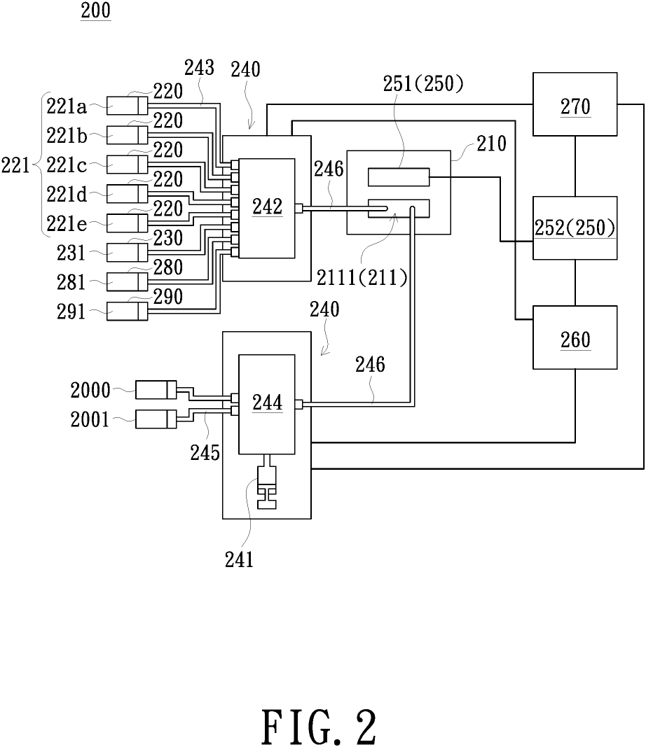

FIG. 2 is a schematic diagram of an apparatus for preparing nucleic acid sequences using an enzyme of one embodiment of the invention. Referring to FIG. 2, an apparatus for preparing nucleic acid sequences using an enzyme 200 of the embodiment includes a reactor 210, a plurality of nucleotide material bottles 220, a deblocking material bottle 230, a liquid delivering device 240, a heater 250, a power supply element 260, and a control module 270. The reactor 210 includes a reaction substrate 211 having a pretreated surface 2111. Each of the nucleotide material bottles 220 is adapted to contain a first reaction solution 221. The deblocking material bottle 230 is adapted to contain a deblocking solution 231. The heater 250 includes, for example, a heating element 251 and a heating control element 252. The apparatus for preparing nucleic acid sequences using an enzyme 200 of the embodiment is similar to the apparatus for preparing nucleic acid sequences using an enzyme 100, and therefore the description of each element will not be repeated hereinafter. The apparatus for preparing nucleic acid sequences using an enzyme 200 of the embodiment further includes, for example, a nucleotide material bottle 220 containing a first reaction solution 221e using a material of a uracil nucleotide, a restriction enzyme material bottle 280, a washing bottle 290, a waste bottle 2000 and a product bottle 2001. The liquid delivering device 240 includes, for example, an extraction device 241, a first switch valve 242, a plurality of first delivering tubes 243, a second switch valve 244, a plurality of second delivering tubes 245, and a plurality of third delivering tubes 246. The restriction enzyme material bottle 280 is adapted to contain a second reaction solution 281 including a restriction enzyme. The washing bottle 290 is adapted to contain a washing solution 291 for washing the pretreated surface 2111 after a reaction to avoid affecting the next reaction. The waste bottle 2000 is adapted to contain the used first reaction solution 221, the used deblocking solution 231, the used second reaction solution 281, and the used washing solution 291. Product bottle 2001 is adapted to contain the prepared nucleic acid sequences.

In the liquid delivering device 240, the plurality of first delivering tubes 243 are connected between the plurality of nucleotide material bottles 220 and the first switch valve 242, between the deblocking material bottle 230 and the first switch valve 242, between the restriction enzyme material bottle 280 and the first switch valve 242, and between the washing bottle 290 and the first switch valve 242. The first switch valve 242 is connected to the pretreated surface 2111 via the third delivering tube 246 and is adapted to switch between the plurality of first delivering tubes 243. The plurality of second delivering tubes 245 are connected between the waste bottle 2000 and the second switch valve 244, and between the product bottle 2001 and the second switch valve 244. The second switch valve 244 is connected to the pretreated surface 2111 via the third delivering tube 246 and is adapted to switch between the plurality of second delivering tubes 245. The extraction device 241 is, for example, a syringe pump or an air pressure valve, but is not limited thereto. A syringe pump is taken as an example in FIG. 2, and the extraction device 241 is connected to the second switch valve 244.

Specifically, taking the delivery of the first reaction solution 221 as an example, when the extraction device 241 extracts, first the first reaction solution 221 of one of the plurality of nucleotide material bottles 220 (that is, one of the first reaction solutions 221a, 221b, 221c, 221d, 221e) is sequentially delivered to the pretreated surface 2111 for reaction via the first delivering tube 243, the first switch valve 242, and the third delivering tube 246. After the reaction is completed, the extraction device 241 continues to extract, so that the first reaction solution 221 on the pretreated surface 2111 is sequentially delivered to the extraction device 241 via the third delivering tube 246 and the second switch valve 244. Next, the second switch valve 244 switches a valve passage to the waste bottle 2000, and the extraction device 241 pushes the first reaction solution 221 to the waste bottle 2000 via the second delivering tube 245. If the reaction is to be carried out with the deblocking solution 231, the first switch valve 242 switches the valve passage to the deblocking material bottle 230, the second switch valve 244 switches the valve passage back to the pretreated surface 2111, and the extraction device 241 starts to extract. After the reaction of the deblocking solution 231 on the pretreated surface 2111 is completed, the deblocking solution 231 is delivered to the waste bottle 2000. When the preparation of the nucleic acid sequences is completed, the second switching valve 244 switches the valve passage to the pretreated surface 2111, and the extraction device 241 continues to extract, so that the nucleic acid sequences on the pretreated surface 2111 are sequentially delivered to the extraction device 241 via the third delivering tube 246 and the second switch valve 244. Next, the second switch valve 244 switches the valve passage to the product bottle 2001, and the extraction device 241 pushes the nucleic acid sequences to the product bottle 2001 via the second delivering tube 245. The above-mentioned manner of use of the liquid delivering device 240 is adapted to the plurality of nucleotide material bottles 220, the deblocking material bottle 230, the restriction enzyme material bottle 280, and the washing bottle 290. It should be noted that the above is only one specific embodiment of the apparatus for preparing nucleic acid sequences using an enzyme 200. The liquid delivering device 240 may include different elements and delivery manners depending on the design architecture.

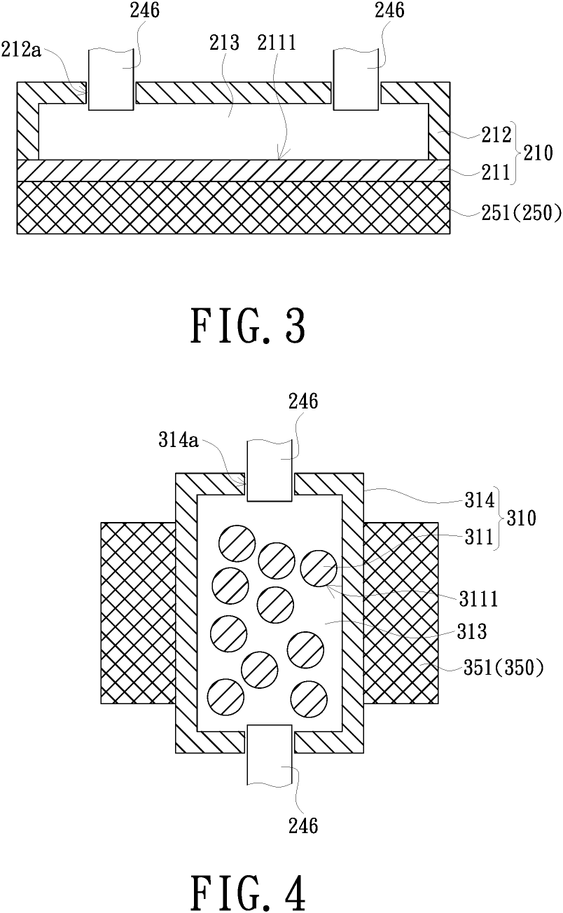

FIG. 3 is a schematic diagram of a combination of a reactor and a heater of one embodiment of the invention. Referring to FIG. 2 and FIG. 3, the heating element 251 of the heater 250 is exemplified by being disposed in the reactor 210 in FIG. 2. Specifically, the reactor 210 includes, for example, the reaction substrate 211 and an upper cover 212, and the reaction substrate 211 and the upper cover 212 are disposed on the heating element 251. The reaction substrate 211 is combined with the upper cover 212 to form a cavity 213, and a surface of the reaction substrate 211 facing the cavity 213 is the pretreated surface 2111. The upper cover 212 has a plurality of through holes 212a adapted to make the third delivering tubes 246 pass through to deliver the first reaction solution 221, the deblocking solution 231, the second reaction solution 281, and the washing solution 291 to the pretreated surface 2111. After the reaction, the first reaction solution 221, the deblocking solution 231, the second reaction solution 281, and the washing solution 291 are delivered to the waste bottle 2000. In other words, the cavity 213 is used as a flow passage. The heating element 251 of the heater 250 heats the reaction substrate 211 directly during the reaction. The embodiment is only one embodiment of the heating, and the invention does not particularly limit the arrangement of the heating element 251.

The apparatus for preparing nucleic acid sequences 200 of the embodiment uses the pretreated surface 2111 of the reaction substrate 211 to prepare nucleic acid sequences, and may simultaneously synthesize a plurality of nucleic acid sequences, thereby improving the efficiency of preparing the nucleic acid sequences.

FIG. 4 is a schematic diagram of a combination of a reactor and a heater of another embodiment of the invention. Referring to FIG. 2 and FIG. 4, the embodiment is another way in which the reactor 310 is combined with the heating element 351 of the heater 350. The reactor 310 includes, for example, the reaction substrate 311 and a case 314. The case 314 has a cavity 313 inside, and the reaction substrate 311 is disposed in the cavity 313. A surface of the case 314 has a plurality of through holes 314a adapted to make the third delivering tubes 246 pass through, and the functions thereof are the same as those described above, and will not be repeated hereinafter. A material of the case 314 is, for example, glass, but is not limited thereto. The reaction substrate 311 of the embodiment is a three-dimensional structure, for example, a spherical shape, and the quantity of the reaction substrate 311 may be plural, and the pretreated surface 3111 is a spherical surface of the reaction substrate 311. The material of the reaction substrate 311 is, for example, superparamagnetic beads, but is not limited thereto. The heating element 351 of the heater 350 surrounds the reactor 310 to heat the reactor 310.

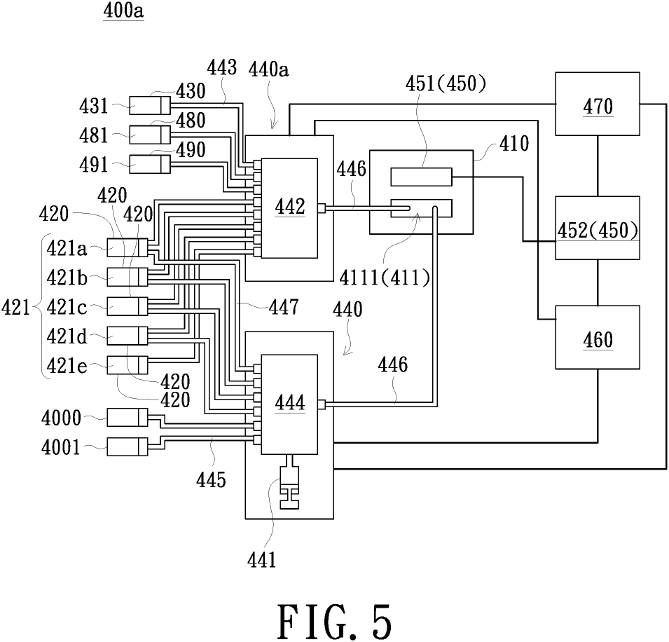

FIG. 5 is a schematic diagram of an apparatus for preparing nucleic acid sequences using an enzyme of another embodiment of the invention. Referring to FIG. 5, an apparatus for preparing nucleic acid sequences using an enzyme 400 of the embodiment includes a reactor 410, a plurality of nucleotide material bottles 420, a deblocking material bottle 430, a liquid delivering device 440, a heater 450, a power supply element 460, and a control module 470, a restriction enzyme material bottle 480, a washing bottle 490, a waste bottle 4000 and a product bottle 4001. The reactor 410 includes a reaction substrate 411 having a pretreated surface 4111. Each of the nucleotide material bottles 420 is adapted to contain a first reaction solution 421. The deblocking material bottle 430 is adapted to contain a deblocking solution 431. The heater 450 includes, for example, a heating element 451 and a heating control element 452. The liquid delivering device 440 includes, for example, an extraction device 441, a first switch valve 442, a plurality of first delivering tubes 443, a second switch valve 444, a plurality of second delivering tubes 445, and a plurality of third delivering tubes 446. The restriction enzyme material bottle 480 is adapted to contain the second reaction solution 481 including a restriction enzyme. The washing bottle 490 is adapted to contain the washing solution 491. The apparatus for preparing nucleic acid sequences using an enzyme 400 of the embodiment is similar in structure and advantages to the apparatus for preparing nucleic acid sequences using an enzyme 200, and therefore the description of each element will not be repeated hereinafter. The difference is only that in the apparatus for preparing nucleic acid sequences using an enzyme 400 of the embodiment, the liquid delivering device 440 further includes, for example, a plurality of fourth delivering tubes 447. The plurality of fourth delivering tubes 447 are respectively connected between the plurality of nucleotide material bottles 420 containing the first reaction solutions 421a, 421b, 421c, 421d and the second switch valve 444. By the plurality of fourth delivering tubes 447, the first reaction solutions 421a, 421b, 421c, 421d after the reaction may be recovered back to the plurality of nucleotide material bottles 420, and may be reused to achieve cost-saving effects.

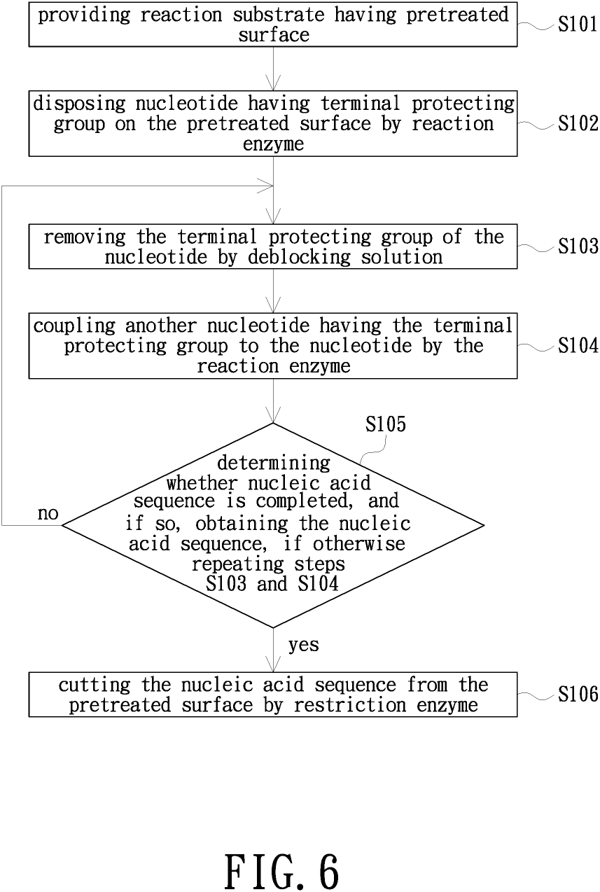

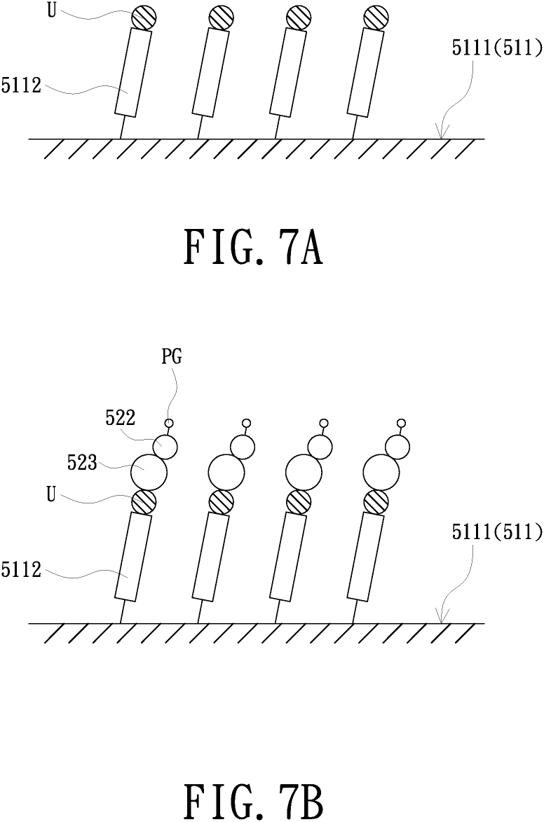

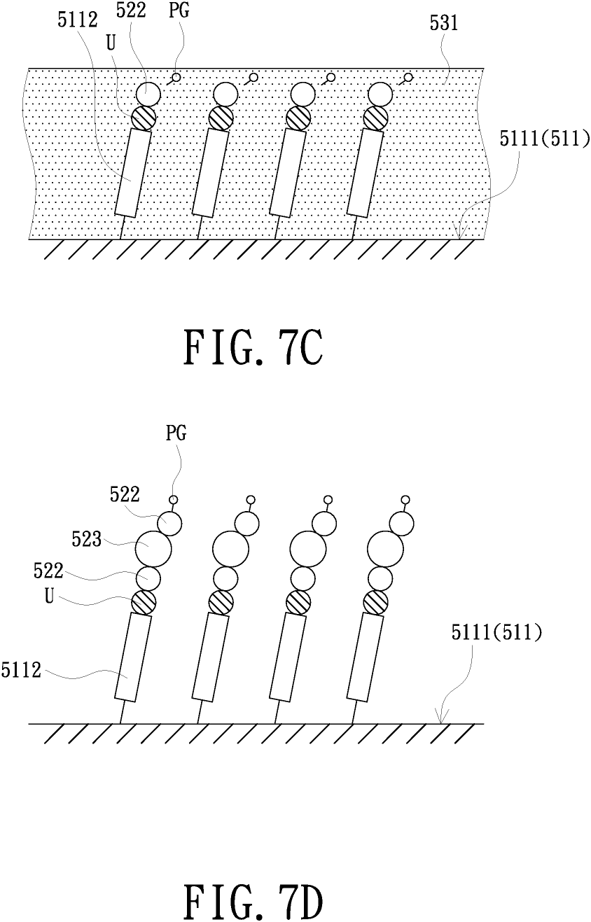

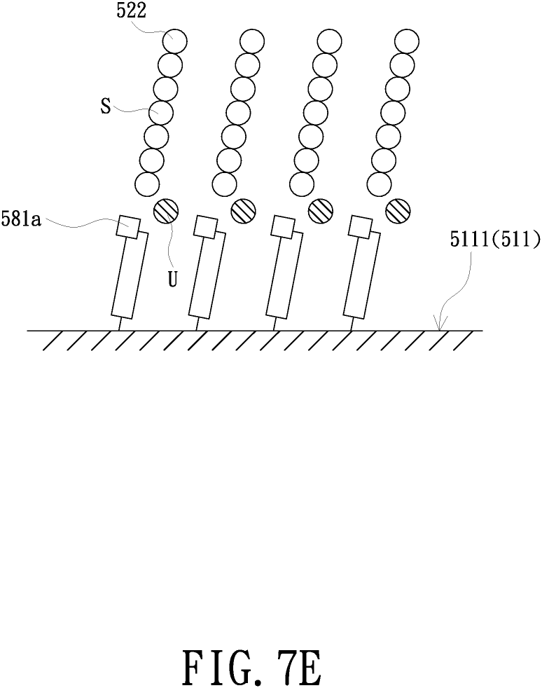

Hereinafter, a method of preparing nucleic acid sequences will be described in further detail, and in the embodiment, synthetic single-stranded deoxyribonucleic acid (ssDNA) sequences is exemplified. FIG. 6 is a schematic flow diagram of a method for preparing nucleic acid sequences using an enzyme of one embodiment of the invention. FIG. 7A to FIG. 7E are schematic diagrams of a reaction of synthesizing nucleic acid sequences of one embodiment of the invention. Referring to FIG. 6 and FIG. 7A first, a method for preparing nucleic acid sequences using an enzyme of the embodiment includes the following steps. Step S101: providing a reaction substrate 511 having a pretreated surface 5111. The pretreated surface 5111 has, for example, a plurality of primers 5112. In the embodiment, the plurality of primers 5112 are, for example, single-stranded DNA, but are not limited thereto. In the embodiment, for example, a uracil nucleotide U is first disposed on the primer 5112, but is not limited thereto. Next, step S102: disposing a nucleotide having a terminal protecting group on the pretreated surface 5111 by a reaction enzyme, and an operating temperature is 45° C. - 105° C., preferably 50° C. -85° C., more preferably 55° C. - 75° C. Specifically, the operating temperature for preparing the nucleic acid sequences is, for example, 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C. or 75° C., but is not limited thereto.

The method of disposing a nucleotide having a terminal protecting group on the pretreated surface includes, for example, disposing a first reaction solution on the pretreated surface 5111, in which the first reaction solution includes the reaction enzyme and the nucleotide having the terminal protecting group. In addition, the method of disposing the first reaction solution on the pretreated surface 5111 includes, for example, delivering the first reaction solution to the pretreated surface 5111 by a liquid delivering device. The first reaction solution is, for example, the first reaction solutions 121, 221, 421 of any of the above embodiments. The liquid delivering device is, for example, the liquid delivering device 140, 240, 440 of any of the above embodiments.

Referring to FIG. 7B, the method of disposing the nucleotide 522 having the terminal protecting group PG on the pretreated surface 5111 includes coupling the nucleotide 522 having the terminal protecting group PG to the uracil nucleotide U on the primers 5112 by the reaction enzyme 523. The reaction enzyme 523 of the embodiment is, for example, a DNA polymerase, particularly a heat stable DNA polymerase, but is not limited thereto. The DNA polymerase includes, for example, family A DNA polymerase, family B DNA polymerase and family X DNA polymerase, and examples thereof include Taq DNA polymerase, archaeal DNA polymerase or thermally stable reverse transcriptase. These DNA polymerases may exhibit better activity at a temperature of 45° C. or higher than the terminal deoxynucleotidyl transferase (TdT) which is conventionally used at 37° C., thereby improving the synthesis efficiency of the nucleic acid sequences. Examples of the terminal protecting group PG include methyl, 2-nitrobenzyl, 3’-O-(2-cyanoethyl), allyl, amine, azidomethyl, tert-butoxy ethoxy (TBE) and the like.

After the step S102 is completed, the reaction enzyme 523 and the unreacted nucleotide 522 having the terminal protecting group PG may be washed away by the washing solution to avoid affecting the subsequent reaction. In the embodiment, the washing solution is, for example, deionized water, but not limited thereto, and the washing solution is, for example, the washing solutions 291, 491 of any of the above embodiments. Next, step S103: removing the terminal protecting group PG of the nucleotide 522 by deblocking solution 531. Referring to FIG. 7C, the deblocking solution 531 may be delivered to the pretreated surface 5111, for example, by the liquid delivering device 140, 240, 440. The function of the deblocking solution 531 is to remove the terminal protecting group PG of the nucleotide 522, so that the nucleotide 522 may be coupled to other nucleotides 522 for synthesis of the nucleic acid sequences. The deblocking solution 531 is classified into, for example, chemical deblocking and enzymatic deblocking depending on different types. Examples of chemical deblocking include such as reducing agents, including DTT (dithiothreitol) or DTE (dithioerythritol). Examples of enzymatic deblocking include phosphatases, including alkaline phosphatase, pyrophosphatase, calf intestinal alkaline phosphatase (CIP) and the like.

After the step S103 is completed, the deblocking solution 531 and the terminal protecting group PG are washed away by the washing solution. Next, step S104: coupling another nucleotide 522 having the terminal protecting group PG to the nucleotide 522 disposed on the pretreated surface 5111 by the reaction enzyme 523. Referring to FIG. 7D, the reaction of step S104 is similar to step S102, except that the nucleotide 522 having the terminal protecting group PG is coupled to the nucleotide 522 that has been previously coupled to the primer 5112. Depending on a length of the designed nucleic acid sequence, step S105 is followed: determining whether a nucleic acid sequence is completed, and if so, obtaining the nucleic acid sequence, if otherwise repeating steps S103 and S104 to continue extending the length of the nucleic acid sequence until the designed nucleic acid sequence is completed.

After the synthesis of the nucleic acid sequences is completed, the method for preparing nucleic acid sequences using an enzyme further includes, for example, step S106: cutting the nucleic acid sequence from the pretreated surface by a restriction enzyme. Referring to FIG. 2 and FIG. 7E, the restriction enzyme material bottle 280 in the apparatus for preparing nucleic acid sequences using an enzyme 200 is adapted to contain the second reaction solution 281 including the restriction enzyme 581a. After the liquid delivering device 240 delivers the second reaction solution 281 to the pretreated surface 5111, the restriction enzyme 581a cuts the synthesized nucleic acid sequence S from the pretreated surface 5111. The nucleic acid sequences S are then collected and delivered to the product bottle 2001 to complete the preparation process of the nucleic acid sequences S. Examples of the restriction enzyme 581a include Uracil DNA Glycosylase (UDG) and Endonuclease VIII, or USER enzyme (NEB #M5508) and a combination thereof. In the embodiment, for example, the restriction enzyme 581a that is cut to the uracil nucleotide U is used. When the nucleic acid sequences are completed, since they are DNA sequences, the DNA sequences may not contain the uracil nucleotide U. For the restriction enzyme 581a, only the previously disposed single uracil nucleotide U may be cut, thereby being able to correctly cut the nucleic acid sequences S. FIG. 7E is a simplified schematic of the cut. The different restriction enzymes 581a will have different cutting sites, and the invention is not particularly limited. The method may also be applied to the apparatus for preparing nucleic acid sequences using an enzyme 400.

In summary, since the apparatus and the method for preparing nucleic acid sequences using an enzyme of the embodiment of the invention use an enzyme to synthesize a nucleic acid sequence, it is less likely to pollute the environment and may reduce the cost compared to the method of chemical synthesis. In addition, the reactor has an operating temperature of 45° C. - 105° C., compared to the conventional use of enzyme at 37° C., the embodiment of the invention may exhibit better activity by using an enzyme at 45° C. or higher, and the synthesis of a plurality of nucleic acid sequences may be carried out simultaneously with the apparatus for preparing nucleic acid sequences using an enzyme of the invention, thereby increasing the efficiency of preparing nucleic acid sequences.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An apparatus for preparing nucleic acid sequences using an enzyme, comprising:
a reactor, comprising a reaction substrate having a pretreated surface;
a plurality of nucleotide material bottles, wherein each of the nucleotide material bottles is adapted to contain a first reaction solution, and the first reaction solution comprises a reaction enzyme and a nucleotide having a terminal protecting group;
a deblocking material bottle, adapted to contain a deblocking solution; and
a liquid delivering device, connected to the reactor, the nucleotide material bottles and the deblocking material bottle, and adapted to deliver the first reaction solution and the deblocking solution to the pretreated surface, wherein the reaction enzyme is adapted to dispose the nucleotide having the terminal protecting group on the pretreated surface, the deblocking solution is adapted to remove the terminal protecting group of the nucleotide,
wherein the reactor has an operating temperature of 45° C. - 105° C.

2. The apparatus for preparing nucleic acid sequences using an enzyme according to claim 1, wherein the reactor has the operating temperature of 50° C. - 85° C.

3. The apparatus for preparing nucleic acid sequences using an enzyme according to claim 2, wherein the reactor has the operating temperature of 55° C. - 75° C.

4. The apparatus for preparing nucleic acid sequences using an enzyme according to claim 1, wherein the reaction enzyme is a DNA polymerase.

5. The apparatus for preparing nucleic acid sequences using an enzyme according to claim 4, wherein the reaction enzyme is family A DNA polymerase.

6. The apparatus for preparing nucleic acid sequences using an enzyme according to claim 4, wherein the reaction enzyme is family B DNA polymerase.

7. The apparatus for preparing nucleic acid sequences using an enzyme according to claim 4, wherein the reaction enzyme is family X DNA polymerase.

8. The apparatus for preparing nucleic acid sequences using an enzyme according to claim 1, wherein the liquid delivering device comprises an extraction device adapted to extract the first reaction solution and the deblocking solution to the pretreated surface.

9. The apparatus for preparing nucleic acid sequences using an enzyme according to claim 8, wherein the extraction device is a syringe pump or an air pressure valve.

10. The apparatus for preparing nucleic acid sequences using an enzyme according to claim 1, wherein the pretreated surface has a plurality of primers adapted to couple the nucleotide having the terminal protecting group.

11. The apparatus for preparing nucleic acid sequences using an enzyme according to claim 1, wherein the deblocking solution comprises a reducing agent or a deblocking enzyme.

12. The apparatus for preparing nucleic acid sequences using an enzyme according to claim 1, further comprising a restriction enzyme material bottle, adapted to contain a second reaction solution comprising a restriction enzyme, wherein the liquid delivering device is further connected to the restriction enzyme material bottle to deliver the second reaction solution to the pretreated surface.

13. The apparatus for preparing nucleic acid sequences using an enzyme according to claim 1, further comprising a heater, connected to the reactor and adapted to heat the reactor.

14. The apparatus for preparing nucleic acid sequences using an enzyme according to claim 1, wherein the liquid delivering device comprises a first switch valve and a plurality of first delivering tubes, the first delivering tubes are connected between the nucleotide material bottles and the first switch valve and between the deblocking material bottle and the first switch valve, the first switching valve is connected to the reactor and adapted to switch between the first delivering tubes.

15. The apparatus for preparing nucleic acid sequences using an enzyme according to claim 1, further comprising a waste bottle and a product bottle, wherein the liquid delivering device comprises a second switch valve and a plurality of second delivering tubes, the second delivering tubes are connected between the waste bottle and the second switch valve and between the product bottle and the second switch valve, the second switching valve is connected to the reactor and adapted to switch between the second delivering tubes.

16. The apparatus for preparing nucleic acid sequences using an enzyme according to claim 1, further comprising a power supply element, adapted to supply power to the liquid delivering device.

17. A method for preparing nucleic acid sequences using an enzyme, comprising:
(1) providing a reaction substrate having a pretreated surface;
(2) disposing a nucleotide having a terminal protecting group on the pretreated surface by a reaction enzyme, and an operating temperature is 45° C. - 105° C.;
(3) removing the terminal protecting group of the nucleotide by a deblocking solution;
(4) coupling another nucleotide having the terminal protecting group to the nucleotide by the reaction enzyme, and an operating temperature is 45° C. - 105° C.; and
(5) determining whether a nucleic acid sequence is completed, and if so, obtaining the nucleic acid sequence, if otherwise repeating steps (3) and (4).

18. The method for preparing nucleic acid sequences using an enzyme according to claim 17, wherein the reactor has the operating temperature of 50° C. - 85° C.

19. The method for preparing nucleic acid sequences using an enzyme according to claim 18, wherein the reactor has the operating temperature of 55° C. - 75° C.

20. The method for preparing nucleic acid sequences using an enzyme according to claim 17, wherein the method of disposing a nucleotide having a terminal protecting group on the pretreated surface comprises disposing a first reaction solution on the pretreated surface, wherein the first reaction solution comprises the reaction enzyme and a nucleotide having the terminal protecting group.

21. The method for preparing nucleic acid sequences using an enzyme according to claim 17, wherein the method of disposing the first reaction solution on the pretreated surface comprises delivering the first reaction solution to the pretreated surface by a liquid delivering device.

22. The method for preparing nucleic acid sequences using an enzyme according to claim 17, wherein the method of adjusting the operating temperature to 45° C. - 105° C. comprises heating the reaction substrate by a heater.

23. The method for preparing nucleic acid sequences using an enzyme according to claim 17, wherein the pretreated surface has a plurality of primers, the method of disposing a nucleotide having a terminal protecting group on the pretreated surface comprises coupling the nucleotide having the terminal protecting group to the primers by the reaction enzyme.

24. The method for preparing nucleic acid sequences using an enzyme according to claim 17, wherein the reaction enzyme is family A DNA polymerase.

25. The method for preparing nucleic acid sequences using an enzyme according to claim 17, wherein the reaction enzyme is family B DNA polymerase.

26. The method for preparing nucleic acid sequences using an enzyme according to claim 17, wherein the reaction enzyme is family X DNA polymerase.

27. The method for preparing nucleic acid sequences using an enzyme according to claim 17, further comprising: (6) cutting the nucleic acid sequence from the pretreated surface by a restriction enzyme.
